(19) United States
(12) Patent Application Publication
(10) Pub. No.: US 2018/0169371 A1
(43) Pub. Date: Jun. 21, 2018

(54) Wearable Non-Contact Qi Emitter

(71) Applicant: Jack Y. Dea, San Diego, CA (US)

(72) Inventor: Jack Y. Dea, San Diego, CA (US)

(21) Appl. No.: 15/380,902

(22) Filed: Dec. 15, 2016

(51) Int. Cl. A61M 21/00; A61N 2/02

(52) U.S. Cl. CPC A61M 21/00; A61M 2021/0055; A61N 2/02

(57) ABSTRACT

A Qi emitting device includes a ferromagnetic core, toroidal wire wrapping about the core, and an oscillator. A powered device further includes a power source and switch. A pendant style device includes a round core and attached cord. A freestanding device includes a frame and stand. A thymus-specific Qi emitting device has a two hertz oscillator and wire wrapping at four turns per inch.

### BACKGROUND

The present invention relates to the technology of non-contact Qi emission. Qi is the Chinese name for the life energy that circulates in the human body. Qi flows freely in a healthy body but is stagnant when the body becomes unhealthy.

The field of Qi emission almost always involves a human emitter, usually a “Qi Gong” practitioner or Qi Gong master. Such a Qi Gong practitioner is not always available. For minor aches and pain, calling on a Qi Gong practitioner may not be worth the associated trouble and expense.

In addition, a person may want to have an energy boost regularly (e.g., every morning) and it is impractical to call upon a Qi Gong practitioner so often.

Therefore there exists a need for a portable Qi emitting device.

### SUMMARY

Some embodiments include a low frequency oscillator driving current into a toroid. The oscillations may be sine waves, triangular waves, or other appropriate periodic waveforms. An influence field of several toroid diameters may be generated by (and surround) the device as a low frequency magnetic field is circulated through the device. A person wearing the device often may feel a tingling or cool breeze sensation near the device. Although the influence field may be perceived (e.g., as a tingling or cool breeze sensation) outside of the device, the device may not produce any measurable electric or magnetic fields.

Specific frequencies may cause effects on specific parts of the body. When a specific part of the body is resonated by Qi, it is stimulated to be more active. In particular, the thymus area may be stimulated by a Qi device of some embodiments that operates at two hertz (Hz). An activated thymus may provide a tremendous health boost for users. The lungs, being in the vicinity of the thymus, may also be stimulated by the Qi emission.

Some wearable embodiments of the Qi emitter device may be capable of producing an influence field of about six inches from the device and may be worn around the neck of a person as a pendant.

The Qi emitter device of some embodiments may include a large rectangular iron core (e.g., two feet by three feet). The influence form such a device may be about six feet. Thus, the influence field of such a device placed in the center of a twelve foot by twelve foot room will influence everyone in the room.

The frequency of such a large device may be selected such that only certain regions of the body are affected. Thus, even though a two Hz influence field from a large rectangular embodiment may flow toward a person's whole body when the person is several feet away, only the thymus area may be affected. When such a device is powered off, the device may remain functional but not directed to specific parts of the body.

The preceding Summary is intended to serve as a brief introduction to various features of some exemplary embodiments. Other embodiments may be implemented in other specific forms without departing from the scope of the disclosure.

### BRIEF DESCRIPTION OF THE SEVERAL VIEWS OF THE DRAWINGS

The exemplary features of the disclosure are set forth in the appended claims. However, for purpose of explanation, several embodiments are illustrated in the following drawings.

FIG. 1 illustrates a front elevation view of a wearable Qi emitter according to an exemplary embodiment;

FIG. 2 illustrates a front elevation view of a large field Qi emitter according to an exemplary embodiment; and

FIG. 3 illustrates a side elevation view of the large field Qi emitter of FIG. 2.

### DETAILED DESCRIPTION

The following detailed description describes currently contemplated modes of carrying out exemplary embodiments. The description is not to be taken in a limiting sense, but is made merely for the purpose of illustrating the general principles of some embodiments, as the scope of the disclosure is best defined by the appended claims.

Various features are described below that can each be used independently of one another or in combination with other features. Broadly, some embodiments generally provide a non-contact device that omits an influence field.

The influence field may be cool and soothing and act similar to a Qi field emitted by a Qi Gong practitioner. The device may include a ferromagnetic core, wiring, and an electronic oscillator. Some embodiments may include the device in a pendant or other wearable item.

The core may be an iron or steel ring with a diameter of a few inches. Square and rectangular (and/or otherwise-shaped) cores may also be used. Larger cores may be used for standalone devices.

A first exemplary embodiment provides a non-contact Qi emitting device comprising: a ferromagnetic core; a wire wrapped around the ferromagnetic core in a toroidal winding; and an electronic oscillator, wherein each of two ends of the wire are terminated at each of two leads of the electronic oscillator.

A second exemplary embodiment provides a Qi emitting device comprising: a ferromagnetic core; a wire wrapped around the ferromagnetic core in a toroidal winding; and an oscillator.

A third exemplary embodiment provides an emitting device comprising: a ferromagnetic core having a toroidal wire wrap; and an oscillator.

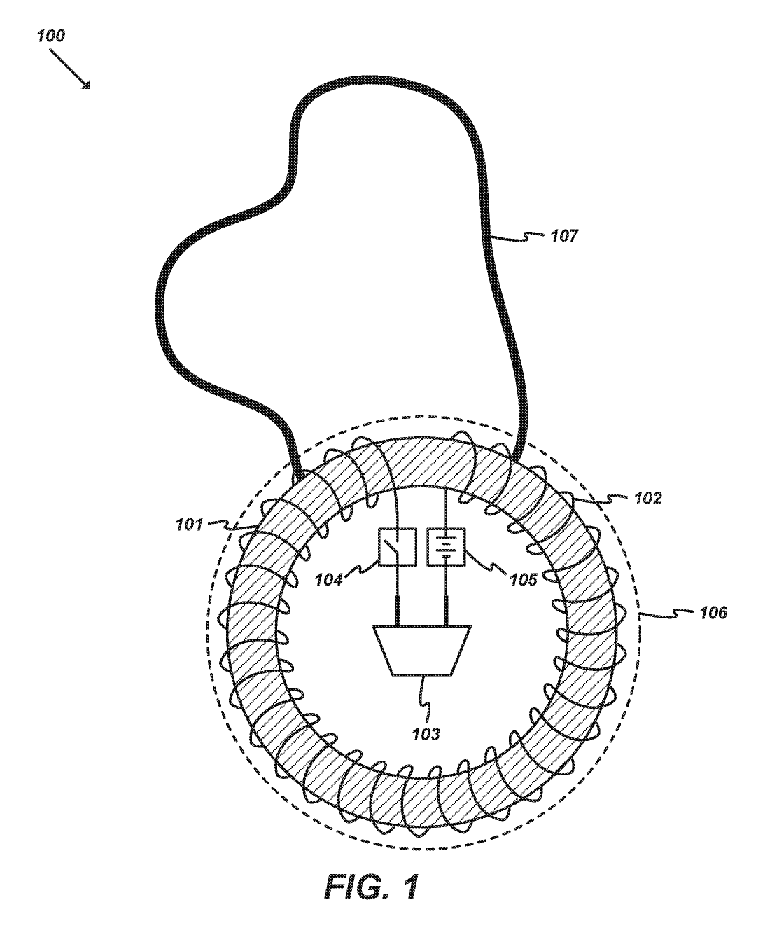

FIG. 1 illustrates a front elevation view of a wearable Qi emitter 100 according to an exemplary embodiment. As shown, the emitter 100 may include a metallic core 101, wire 102, oscillator 103, switch 104, battery 105, housing 106, and cord 107.

The core 101 may be made of iron and may be wrapped in a toroidal fashion by wire 102. Typically, the wire may be wound at four turns per inch of core. However, the number of turns of wire is not critical.

The electronic oscillator 103 may operate at two Hz (and/or other appropriate frequencies) and have two leads, connected in series with wire 102. When such a device 100 is turned off, the device may still function but not target specific body parts.

The switch 104 may be an on-off double pole double throw (DPDT) switch that may connect the wire 102 to one lead of the oscillator 103. In the “off” position, the circuit may be open. In the “on” position, the circuit may be closed.

Battery 105 may power the emitter 100. In some embodiments, the emitter may include various charging connectors (e.g., USB connectors) or elements (e.g., solar cells) that may be used to provide charging power to the battery 105 and/or to power the device 100 directly.

Housing 106 and/or coverings may be used to at least partially enclose components 101-105 (or portions thereof). Such housings may include various materials (e.g., plastic, fabric, metal, leather, glass, etc.). The housing 106 may generally have a smooth, continuous surface that may expose various elements of the device (or portions thereof). For instance, the housing may include a charging port or connector and may provide access to a manipulator for switch 104.

The cord 107 (or lanyard, strap, etc.) may connect to the housing and enable the user to wear the emitter 100 around the neck.

One of ordinary skill in the art will recognize that while the device of FIG. 1 has been described with reference to various details, different embodiments may implemented in various different ways without departing from the scope of the disclosure. For instance, some embodiments may be implemented as wearable devices that are able to be strapped to a wrist or waist (similar to a watch or fanny-pack, for instance). As another example, some embodiments may be embedded into a cap, hat, belt, and/or other wearable clothing and/or accessories. Some embodiments may be passive (and/or may operate passively), whereby no power supply is connected or the device is in an off state. Such a configuration may operate as a generic emitter, without special effect toward the thymus or lungs.

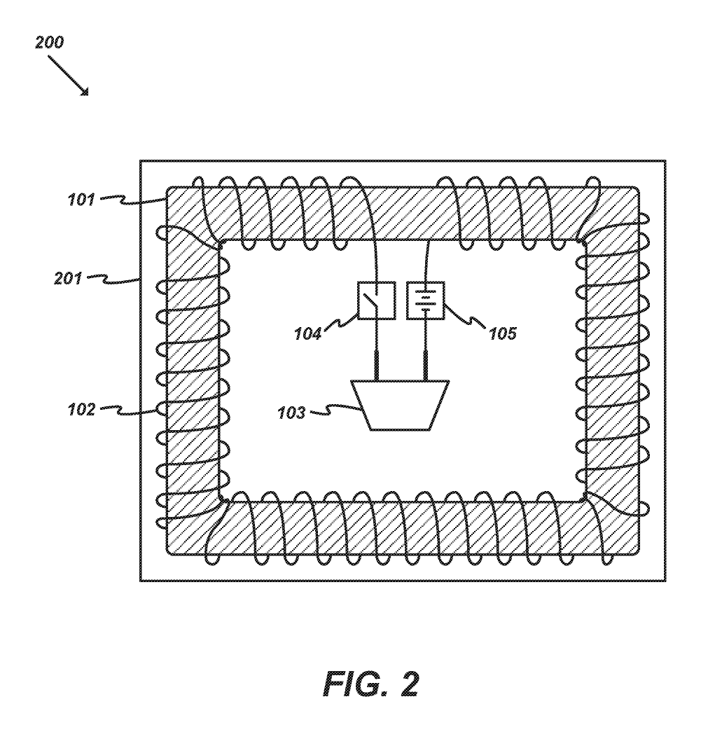

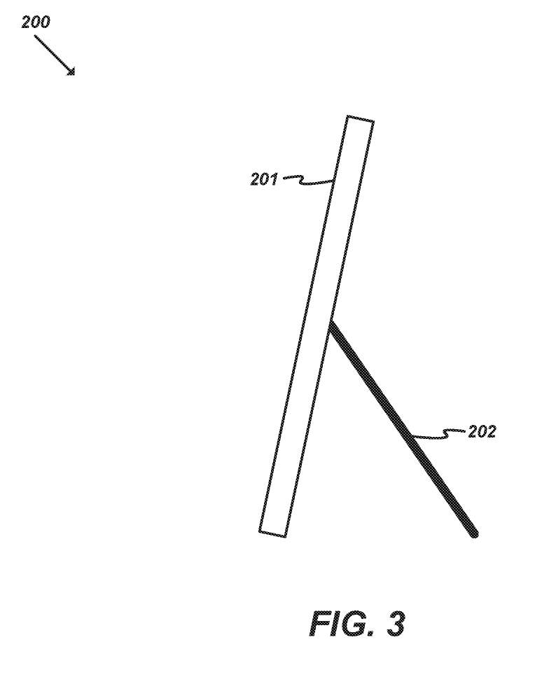

FIG. 2 illustrates a front elevation view of a large field Qi emitter 200 according to an exemplary embodiment. FIG. 3 illustrates a side elevation view of the large field Qi emitter 200.

As shown, the components 101-105 may be similar to those described above in reference to emitter 100, with the shape of the core 101 being rectangular in this example. In addition, the emitter 200 may include a frame 201 and stand 202. The frame may be made from various appropriate rigid materials while the stand 202 may be made from wire and/or other appropriate materials.

During operation, the wearable Qi emission device 100 may be worn around the neck of the user. The switch may be placed in an on position for stimulation of the lungs and thymus. Qi emitting device 200 may be stood on a table or other appropriate support a few feet from one or more users.

The Qi emitting devices 100 and 200 may be used to supplement Qi sessions from Qi practitioners. Often times, it is not practical to call upon a Qi practitioner, and the Qi emitting device can act as a substitute. Generally, sitting in the influence field of the Qi emitting device is similar to being in the influence of a Qi practitioner. Typically, the user feels his or her Qi circulation strengthen.

The foregoing relates to illustrative details of exemplary embodiments and modifications may be made without departing from the scope of the disclosure as defined by the following claims.

I claim:

1. A non-contact Qi emitting device comprising:
a ferromagnetic core;
a wire wrapped around the ferromagnetic core in a toroidal winding; and
an electronic oscillator,
wherein each of two ends of the wire are terminated at each of two leads of the electronic oscillator.

2. The non-contact Qi emitting device of claim 1, wherein the ferromagnetic core comprises iron.

3. The non-contact Qi emitting device of claim 1, wherein the electronic oscillator operates at a frequency of two Hz.

4. The non-contact Qi emitting device of claim 1 further comprising a battery and an on-off switch that connects the electronic oscillator in series with the battery.

5. The non-contact Qi emitting device of claim 1, wherein non-contact Qi emitting device is enclosed in a housing connected to a cord able to be worn around a neck of a user.

6. The non-contact Qi emitting device of claim 1 further comprising an enclosure and a stand.

7. The non-contact Qi emitting device of claim 1 further comprising a strap.

8. The non-contact Qi emitting device of claim 1, wherein the non-contact Qi emitting device is embedded into a hat or cap.

9. A Qi emitting device comprising:
a ferromagnetic core;
a wire wrapped around the ferromagnetic core in a toroidal winding; and
an oscillator.

10. The Qi emitting device of claim 9 further comprising:
a power source; and
an on-off switch.

11. The Qi emitting device of claim 10, wherein the power source comprises a rechargeable battery.

12. The Qi emitting device of claim 9, wherein the ferromagnetic core is circular.

13. The Qi emitting device of claim 12, wherein the ferromagnetic core has a diameter of two to three inches.

14. The Qi emitting device of claim 9 further comprising:
a housing that at least partially encloses the ferromagnetic core, the wire, and the oscillator; and
a cord coupled to the housing.

15. An emitting device comprising:
a ferromagnetic core having a toroidal wire wrap; and
an oscillator.

16. The emitting device of claim 15, wherein the toroidal wire wrap comprises four turns per linear inch.

17. The emitting device of claim 15, wherein the oscillator operates at two hertz.

18. The emitting device of claim 15, further comprising a battery and an on-off switch, wherein the toroidal wire wrap, battery, and on-off switch are connected in series.

19. The emitting device of claim 15 further comprising an enclosure and attached stand.

20. The emitting device of claim 15, wherein the ferromagnetic core comprises iron.
